(19) United States
(12) Patent Application Publication
(10) Pub. No.: US 2010/0152129 A1
(43) Pub. Date: Jun. 17, 2010

(54) Novel synergistic combination of gemcitabine with P276-00 or P1446A in treatment of cancer

(76) Inventors: Periyasamy Giridharan, Mumbai (IN); Debarshi Chakrabarti, Mumbai (IN); Amit Khanna, Mumbai (IN); Urvi Ved, Mumbai (IN); Asha Almeida, Mumbai (IN); Somesh Sharma, Mumbai (IN); Muralidhara Padigaru, Mumbai (IN); Arun Balakrishnan, Mumbai (IN)

Correspondence Address: KAUSALYA SANTHANAM, 60 Whittier Road, New Haven, CT, 06515, US

(21) Appl. No.: 12/653,012

(22) Filed: Dec. 7, 2009

Related U.S. Application Data
(63) Continuation-in-part of application No. 12/383,713, filed on Mar. 26, 2009.

(30) Foreign Application Priority Data
Mar. 31, 2008 (IN) 699/MUM/2008

(51) Int. Cl. A61K 31/7068; C12Q 1/68; A61P 35/00

(52) U.S. Cl. 514/49; 435/6

(57) ABSTRACT

Synergistic combinations of gemcitabine with P276-00 or P1446A and their use in the treatment of cancer are disclosed. The invention further describes novel and unique gene signatures comprising gene markers used to monitor the drug response in a subject treated with the said combinations.

### RELATED APPLICATION

This application is a Continuation-in-Part (CIP) of U.S. application Ser. No. 12/383, 713 filed 27 Mar. 2009, which takes priority from Indian Provisional Application ‘Novel Synergistic Combination of Gemcitabine with P276-00 or P1446A in Treatment of Cancer’ filed 31 Mar. 2008, which is incorporated herein in its entirety.

### FIELD OF INVENTION

The present invention relates to synergistic combinations of Gemcitabine with P276-00 or P1446A and their use in the treatment of cancer. The present invention particularly relates to a novel gene signature comprising drug response markers used to monitor the drug response in a subject undergoing treatment for cancer with the said combinations.

### BACKGROUND

It is increasingly being realized that effective treatment of cancer not only requires efficient drugs but also optimal use of specific combination of anti-cancer drugs. During the past 25 years, about 10% to 90% cure rate has been achieved in case of acute lymphoblastic leukemia with the optimization and combination of existing drugs. Combination therapy is thus increasingly being looked at as a better alternative to monotherapy in the treatment of cancer. The sequential or simultaneous use of two or more agents is termed as combination therapy.

Various combinations of chemotherapeutic agents have been discussed in prior art. W02004041308 discloses a combination of CDK inhibitor with Gemcitabine for use in the treatment of cancer. Although the disclosure suggests additive effect, a synergistic effect in using the combination is not very clear. The CDK inhibitor preferably used in said disclosure is Roscovitine. U.S. Pat. No. 7,294,332 describes a combination of temozolomide and IFN a in the treatment of malignant melanoma. In this context, the applicants have previously filed an application W02008139271, which provides a synergistic combination of a CDK inhibitor which is a flavone compound and a cytotoxic antineoplastic agent which is incorporated herein by reference in its entirety.

Gene expression signature for a set of 14 predictor genes is used as a measure of efficacy in patients suffering from colorectal cancer and receiving combination therapy of leucovorin, fluorouracil, and irinotecan (FOLFIRI). The accuracy of prediction in this study was 95% and could be used as decision tool to assist oncologist in selecting colorectal patients for FOLFIRI chemotherapy (Rio et al, J of Clin Oncol 2007; 25 (7); 773-780).

In another study, Li et al, demonstrated that in prostate cancer, docetaxel and estramustine combination treatment directly and indirectly caused changes in the expression of many genes that are critically involved in the control of cell proliferation, apoptosis, transcription, translation, oncogenesis, angiogenesis, metastasis, and drug resistance (Mol Cancer Ther 2005; 4: 389-98). A report by Cheok et al provides molecular insights into the synergy of drug combination for purine antagonist mercaptopurine and dihydrofolate reductase inhibitor methotrexate in leukemia patients using microarray-based gene expression profiling for unique gene signatures for the combination (Nature Genetics 2003; 34:85-90).

Christoph et al (Clinical Cancer Research 2001;7, 2527-2536) showed that Flavopiridol potentiates gemcitabine-induced apoptosis in a sequence-dependent manner in pancreatic (Capan-2), as well as colorectal (HCT-116) and gastric cancer (MKN-74 and SK-GT-5) cell lines. Sequential treatment of Gemcitabine followed by Flavopiridol results in a 10-15-fold increase of apoptotic rates of these adenocarcinoma cell lines, as against treatment with Gemcitabine alone. This is greater than the potential of Flavopiridol to increase induction of apoptosis in mitomycin- and paclitaxel-treated gastric (MKN-74) and breast (MDA-MB-468 and MCF-7) cancer cells.

Fischer and Gianella-Borradori (Expert Opinion on Investigational Drugs 2003;12:955-70) demonstrated that the administration of Gemcitabine in combination with roscovitine produces enhanced cytotoxicity as compared to either drug administrated alone indicating the synergistic interaction between the two components.

The present invention is based on the applicant's observation that Gemcitabine in combination with P276-00 or P1446A shows marked synergistic anti-tumor effect as compared to the independent use of these agents.

Gemcitabine (2′, 2′-difluorodeoxycytidine) is currently being marketed as Gemzar® by Eli Lilly. It is a nucleoside analogue of deoxycytidine, which was first disclosed in U.S. Pat. No. 4,808,614 and U.S. Pat. No. 5,464,826. Gemcitabine has been indicated as the first-line therapy for locally advanced (nonresectable Stage II or III) or metastatic (Stage IV) adenocarcinoma of the pancreas. Gemcitabine has also been indicated as a second-line therapy for patients who have previously been treated with fluorouracil.

Cyclin-dependant kinase (CDK), a class of genes involved in cell cycle pathway is emerging as targets in cancer drug discovery. Several CDK inhibitors are currently undergoing clinical evaluation either as a single agent or in combination with other anti-cancer drugs. Flavones are a series of novel compounds that exhibit significant specific activity against CDKs especially CDK4. P276-00, a flavone compound is (+)-trans-2-(2-Chloro-phenyl)-5,7 -dihydroxy-8-(2-hydroxy-methyl-1-methyl-pyrrolidin-3-y1)-chromen-4-one hydrochloride with potent anti-CDK4 activity and is described in U.S. Pat. No. 7,271,193 which is herein incorporated by reference in its entirety. CDK4 catalyzes the phosphorylation and inhibition of retinoblastoma (RB) protein, which is a negative regulator of cell cycle. Phosphorylation of RB leads to uncontrolled cell proliferation and induces tumorigenesis in cells. P276-00 was found to be more selective for CDK4-D1, CDK1-B, and CDK9 -T1, as compared with other CDKs, and less selective for non-CDK kinases. It showed potent antiproliferative effects against various human cancer cell lines as demonstrated by both in vitro and in vivo experimental conditions. It is also observed that P276-00 induces a significant down-regulation of cyclin D1, CDK4 and CDK4 -specific pRB Ser(780) phosphorylation. The compound also induced apoptosis in human promyelocytic leukemia (HL-60) cells, as evidenced by the induction of caspase-3 and DNA ladder studies.

P1446A is (+)-trans-3-[2[(2-Chloro-4-trifluoromethyl-phenyl)-5,7-dihydroxy-8-(2-hydroxymethyl-1-methyl-pyrrolidin-3-yl)-chromen-4-one hydrochloride. Its mode of action reveals that it is a potent inhibitor of CDK4-D1, CDK1-B and CDK9-T and is described in WO2007148158 which is incorporated herein by reference in its entirety.

The present invention provides a synergistic combination of Gemcitabine with P276-00 or P1446A. The invention also provides a novel and unique gene signature comprising gene markers for monitoring drug response in patients undergoing cancer treatment with the said combinations.

### SUMMARY

The present invention relates to pharmaceutical compositions comprising combination of Gemcitabine with P276-00 or P1446A. The said combinations are used in the treatment of cancer. It further provides a novel gene signature comprising gene markers used to monitor the drug response in a subject administered with the aforementioned combinations.

In one aspect, the invention provides a method of treating cancer in a patient comprising administering the combinations of Gemcitabine with P276-00 or P1446A. The types of cancer treated with these combinations are pancreatic cancer, lung cancer, colorectal carcinoma and head and neck cancer.

The invention provides a gene signature comprising at least two gene markers for monitoring the drug response in a patient administered with the composition of Gemcitabine with P276-00 or P1446A.

The gene signature for the combination of Gemcitabine and P276-00 comprises at least two gene markers selected from the group consisting of SNX7, FA38A, DNAI1, RRM2, CDK8, DLG5, FGF5, MKKS, HELLS, PPIL4, SLC19A2, ID1, DICER1, TMPRSS3 and HIST1H2BO, BAX, Cytochrome C, Caspase-3, pAKT, pRB, CyclinD1, MMP-1, VEGF, CDC25B, P21, P14ARF and PTN. Furthermore, in patients administered with the combination of Gemcitabine and P276-00, up regulation is observed in drug response markers SNX7, FA38A , DNAI1, BAX, Cytochrome C and Caspase-3 and down regulation is observed in RRM2, CDK8, DLG5, FGF5, MKKS, HELLS, PPIL4, SLC19A2, ID1, DICER1, TMPRSS3, HIST1H2BO, pAKT, pRB, CyclinD1, MMP-1, VEGF, CDC25B, P21, P14ARF and PTN.

In another aspect, the invention provides a gene signature for monitoring drug response in a subject administered with the combination of Gemcitabine and P1446A comprising at least two gene markers selected from the group consisting of P21, REV3L, FGF5, PTK7, POLH, P27 and SSTR2. Upon administration of the combination Gemcitabine and P1446A, there is upregulation of the said markers.

In yet another aspect, the invention provides a method of monitoring the drug response in a patient suffering from cancer and treated with a combination of Gemcitabine and P276-00, comprising detection of gene signature with at least two markers selected from the group consisting of SNX7, FA38A, DNAI1, RRM2, CDK8, DLG5, FGF5, MKKS, HELLS, PPIL4, SLC19A2, ID1, DICER1, TMPRSS3 , HIST1H2BO, BAX, Cytochrome C, Caspase-3, pAKT, pRB, CyclinD1, MMP-1, VEGF, CDC25B, P21, P14ARF and PTN.

The invention also provides a method of monitoring the drug response in a patient suffering from cancer and treated with a combination of Gemcitabine and P1446A, comprising detection of gene signature with at least two gene markers selected from the group consisting of P21, REV3L, FGF5, PTK7, POLH, P27 and SSTR2.

### BRIEF DESCRIPTION OF FIGURES

FIG. 1: Effect of sequentially administered combination of Gemcitabine with P276-00 on Panc-1 cells using propidium iodide based fluorescence cytotoxicity assay.

FIG. 2: Effect of sequentially administered combination of Gemcitabine with P276-00 on Panc-1 cells using Flow cytometry.

FIG. 3: Effect of sequentially administered combination of Gemcitabine with P1446A on Panc-1 cells using CCK-8 cytotoxicity assay.

FIG. 4: Comparison of the drug induced cytotoxicity in Panc-1 cells at various time points after drug treatment.

FIG. 5: Comparison of the drug induced gene expression profile in Panc-1 cells.

FIG. 6: Drug induced Phospho AKT expression in Panc-1 cells.

FIG. 7: Drug induced Phospho RB expression in Panc-1 cells.

FIG. 8: Drug induced Pleiotrophin (PTN) expression in Panc-1 cells.

FIG. 9: Drug induced CDC25B expression in Panc-1 cells.

FIG. 10: Drug induced CyclinD1 expression in Panc-1 cells.

FIG. 11: Drug induced VEGF expression in Panc-1 cells.

FIG. 12: Drug induced protein expression in Panc-1 cells for various proteins (a) Drug induced protein expression in Panc-1 cells for BCL2 (b) Drug induced protein expression in Panc-1 cells for P27 protein (c) Drug induced protein expression in Panc-1 cells for MMP1 protein.

FIG. 13: Drug efficacy score at various time intervals for cancer related proteins in Panc-1 cells.

### DETAILED DESCRIPTION OF THE INVENTION

Gemcitabine, a known therapeutic agent for cancer shows synergistic tumoricidal effect with P276-00 or P1446A as compared to the compounds administered individually. The present invention discloses a novel gene signature comprising at least two gene markers for monitoring drug response in a subject administered with the combination of Gemcitabine with P276-00 or P1446A.

As has been seen previously (W02008139271 incorporated herein in its entirety), the combination of Gemcitabine and P276-00 or P1446A described in the instant disclosure shows a synergistic anti-cancer effect as compared to the agents given individually. In addition, the synergy established by the combination of Gemcitabine and P276-00 or P1446A is higher than the combination of Gemcitabine with other known CDK inhibitors of therapeutic importance. This synergistic effect of the aforementioned synergistic combinations forms the basis of the invention.

Gemcitabine (2′-deoxy-2′, 2′-difluorocytidine) is a nucleoside analogue of deoxycytidine. It exhibits cell phase specificity, primarily killing cells undergoing DNA synthesis (S-phase) and also blocking the progression of cells through the G1/S-phase boundary. Gemcitabine is metabolized intracellularly by nucleoside kinases to the active diphosphate (dFdCDP) and triphosphate (dFdCTP) nucleosides. Its cytotoxic effect can be attributed to the inhibition of DNA synthesis as a result of the combined actions of the diphosphate and triphosphate nucleosides. More specifically, Gemcitabine diphosphate inhibits ribonucleotide reductase, which is responsible for catalyzing the reactions that generate the deoxynucleoside triphosphates for DNA synthesis. Inhibition of this enzyme by the diphosphate nucleoside causes a reduction in deoxynucleotide concentrations, for example dCTP. Furthermore, Gemcitabine triphosphate competes with dCTP for incorporation into DNA. The subsequent reduction in the intracellular concentration of dCTP enhances the incorporation of Gemcitabine triphosphate into DNA (self potentiation). Gemcitabine exhibits antitumour activity, particularly against ovarian, pancreatic and lung cancers. Gemcitabine finds use in the first-line therapy for locally advanced (nonresectable Stage II or III) or metastatic (Stage IV) adenocarcinoma of the pancreas. Patients previously treated with fluorouracil use Gemcitabine as a second-line therapeutic agent.

P276-00 is (+)-trans-2-(2-Chloro-phenyl)-5,7-dihydroxy-8-(2-hydroxy-methyl-1-methyl-1pyrrolidin-3-yl)-chromen-4-one hydrochloride, a flavone compound with anti-CDK4 activity and is disclosed in U.S. Pat. No. 7,271,193. The compound finds use in antiproliferative therapies for diseases characterized by excessive cell growth such as cancers, cardiovascular abnormalities, nephrological disorders, psoriasis, Alzheimer's disease, immunological disorders involving unwanted proliferation of leukocytes, restenosis and other proliferative smooth muscle disorders, viral infections, and mycotic infections. P1446A, (+)-trans--3-[2[(2-Chloro-4-trifluoromethyl-phenyl)-5,7-dihydroxy-8-(2-hydroxymethyl-1-methyl-pyrrolidin-3-yl)-chromen-4-one hydrochloride, is a novel, potent inhibitor of CDK4-D1, CDK1-B and CDK9-T (See WO2007148158). Pre-clinical data from human cancer cell lines has shown that P1446A halts transcriptional elongation, promotes apoptosis and arrests cell cycle progression at G1 and G2 phases of cell cycle. In vitro studies with a variety of cancer cell lines suggest that P1446A effectively inhibits proliferation of and induces cytotoxicity in both Cisplatin sensitive and resistant cells without any significant cytotoxicity to normal human peripheral blood mononuclear cells. P1446A induces significant down regulation of Cyclin DI and CDK4 specific retinoblastoma protein (pRb) ser780 phosphorylation, induced p53 and reduced the levels of the anti-apoptotic protein Bcl-2. P1446A also demonstrated good oral bioavailability in the preclinical studies. Upon oral administration, it demonstrated significant tumor growth inhibition in xenograft models of colon cancer (HCT-116, SW-480) and non-small cell lung cancer (H-460) in SCID mice. By virtue of its specific action against CDK4-D1, CDK1-B and CDK9-T, P1446A has the potential to have potent effects on cell cycle arrest while avoiding the unwanted effects associated with more non-specific CDK inhibitors. In addition, P1446A being an oral therapeutic has an additional advantage of increased compliance among cancer patients.

In another embodiment, the invention provides a method of treating cancer by administering combinations of Gemcitabine with P276-00 or P1446A. The types of cancer treated with the said combinations include but are not limited to pancreatic cancer, lung cancer, colorectal carcinoma and head and neck cancers. In a preferred embodiment, the said combinations are used to treat pancreatic cancer.

In yet another embodiment, the invention provides a dosage range of the combinations used to administer to the patients suffering from cancer. The dosage varies between a final concentration range of 0.1 nM-30 nM for Gemcitabine and 60-960 nM for P276-00. In case of combination of Gemcitabine with P1446A, the dosage varies between a final concentration of 3 nM-10000 nM for Gemcitabine and 1 nM-10000 nM for P1446A.

Various possible combinations of the agents in the aforementioned dosage ranges are used in the invention.

The invention also provides a method of administering the said combinations. The method involves administering the combinations either simultaneously or sequentially. The method of administration preferably is sequential wherein Gemcitabine is first administered followed by the administration of P276-00 or P1446A. Gemcitabine is administered as a 30 min bolus infusion while P276-00 is administered intravenously or as a bolus infusion for 30 mins. P1446A is administered orally.

In one embodiment, the present invention provides a dosing schedule of the combinations. The combination of Gemcitabine with P276-00 is administered in the following steps; Gemcitabine is first administered for 0-24 hrs followed by sequential administration of P276-00 for 24-96 hrs. The combination of Gemcitabine with P1446A is administered in the following steps; Gemcitabine is first administered for 0-24 hrs followed by sequential administration of P1446A for 24-72 hrs.

The present invention provides a novel gene signature for monitoring the drug response in a patient suffering from cancer administered with the combinations of Gemcitabine and P276-00 or Gemcitabine and P1446A. Method of monitoring the drug response following administration of Gemcitabine and P276-00 or Gemcitabine and P1446A is also encompassed in the invention.

Microarray-based gene signatures provide valuable information regarding the cellular function and can be effectively used in determining the drug response in patients subsequent to exposure to combination of therapeutic agents. (Nature Genetics 2003; 34:85-90).

The gene signature of the present invention used to monitor the drug response in a patient undergoing treatment for cancer administered with the combination of Gemcitabine and P276-00 comprises of at least two gene markers selected from the group consisting of SNX7, FA38A, DNAI1, RRM2, CDK8, DLG5, FGF5, MKKS, HELLS, PPIL4, SLC19A2, ID1, DICER1, TMPRSS3, HIST1H2BO, BAX, Cytochrome C, Caspase-3, pAKT, pRB, CyclinD1, MMP-1, VEGF, CDC25B, P21, P14ARF and PTN. Amongst these markers SNX7, FA38A, DNAI1, BAX, Cytochrome C and Caspase-3 are up regulated while RRM2, CDK8, DLG5, FGF5, MKKS, HELLS, PPIL4, SLC19A2, ID1, DICER1, TMPRSS3, HIST1H2BO, pAKT, pRB, CyclinD1, MMP-1, VEGF, CDC25B, P21, P14ARF and PTN are down regulated in patients administered with the combination discussed above.

The above disclosed drug response markers are detailed below:

Sorting Nexin 7 (SNX7): This gene encodes a member of the sorting nexin family. Sorting nexins are proteins involved in protein trafficking in the endosomes and the vesicular micro-tubular structures in the cytoplasm. Two isoforms of the sorting nexin family (SNX1 & SNX2) have been demonstrated to play a functional role in localization of the endogenous EGFR in the endosomes of colon cancer cells. Deletion mutants for SNX1 have proved to down regulate the endogenous EGFR expression. Although there is no current evidence to prove the role of SNX7 in the pathology of cancer, our data could help in identification of new role for SNX7 in pancreatic cancer disease biology.

Dynein, axonemal, intermediate chain 1 (DNAI1): The inner and outer arm dyneins, which bridge between the doublet microtubules in axonemes, are the force-generating proteins responsible for the sliding movement in axonemes. The intermediate and light chains, thought to form the base of the dynein arm, help mediate attachment and may also participate in regulating dynein activity. This gene encodes an intermediate chain dynein, belonging to the large family of motor proteins. Mutations in this gene result in abnormal ciliary ultrastructure and function associated with primary ciliary dyskinesia (PCD) and Kartagener syndrome.

Ribonucleotide reductase M2 polypeptide (RRM2): Ribonucleotide reductase catalyzes the formation of deoxyribonucleotides from ribonucleotides. It is composed of two non-identical subunits, proteins M1 and M2. Synthesis of M2 is regulated in a cell-cycle dependent fashion. Activity of this enzyme, which catalyses conversion of ribonucleotide 5′-diphosphates to their 2′-deoxynucleotides, is modulated by levels of its M2 subunit (RRM2). The present invention reveals that RRM2 overexpression is associated with Gemcitabine chemo resistance in pancreatic adenocarcinoma cells, and that suppression of RRM2 expression using RNA interference mediated by small interfering RNA (siRNA) enhances Gemcitabine-induced cytotoxicity in vitro.

Cyclin-dependent kinase 8 (CDK8): The protein encoded by this gene is a member of the cyclin-dependent protein kinase (CDK) family and is known to be an important regulator of cell cycle progression. This kinase and its regulatory subunit cyclin C are components of the RNA polymerase II holoenzyme complex, which phosphorylates the carboxy-terminal domain (CTD) of the largest subunit of RNA polymerase II. This kinase has also been shown to regulate transcription by targeting the CDK7/cyclin H subunits of the general transcription initiation factor IIH (TFIIH), thus providing a link between the ‘Mediator-like’ protein complexes and the basal transcription machinery. Furthermore, increased binding of CDK8 to p53 target genes correlates positively with transcriptional strength and CDK8 functions as a coactivator within the p53 transcriptional program.

Discs Large Homolog 5 (DLG5): This gene encodes a member of the family of discs large (DLG) homologs, a subset of the membrane-associated guanylate kinase (MAGUK) super family. The protein encoded by this gene localizes to the plasma membrane and cytoplasm, and interacts with components of adherens junctions and the cytoskeleton. It is proposed to function in the transmission of extracellular signals to the cytoskeleton and in the maintenance of epithelial cell structure.

Fibroblast Growth Factor 5 (FGF5): The protein encoded by this gene is a member of the fibroblast growth factor (FGF) family. FGF family members possess broad mitogenic and cell survival activities, and are involved in a variety of biological processes, including embryonic development, cell growth, morphogenesis, tissue repair, tumor growth and invasion. This gene was identified as an oncogene, which confers transforming potential when transfected into mammalian cells.

McKusick-Kaufman syndrome protein (MKKS): This gene encodes a protein with sequence similarity to the chaperonin family. The encoded protein may have a role in protein processing in limb, cardiac and reproductive system development. Mutations in this gene have been observed in patients with Bardet-Biedl syndrome type 6 and McKusick-Kaufman syndrome.

Peptidyl Prolyl Isomerase Like 4 (PPIL4): This gene is a member of the cyclophilin family of peptidylprolyl isomerases. The cyclophilins are a highly conserved family, members of which play an important role in protein folding, immunosuppression by cyclosporin A, and infection of HIV-1 virions.

Helicase, Lymphoid-Specific (HELLS): This gene encodes a lymphoid-specific helicase. Other helicases function in processes involving DNA strand separation, including replication, repair, recombination, and transcription. This protein is thought to be involved with cell proliferation and may play a role in leukemogenesis.

Inhibitor of DNA binding 1 (ID1): The protein encoded by this gene is a helix-loop-helix (HLH) protein that can form heterodimers with members of the basic HLH family of transcription factors. The encoded protein has no DNA binding activity and therefore can inhibit the DNA binding and transcriptional activation ability of basic HLH proteins with which it interacts. This protein may play a role in cell growth, senescence, and differentiation. ID-1 is suggested as an oncogene and is reported to promote cell proliferation, invasion, and survival in several types of human cancer cells through multiple signaling pathways.

Dicer 1 Ribonuclease Type III (DICER1): This gene encodes a protein possessing an RNA helicase motif containing a DEXH box in its amino terminus and an RNA motif in the carboxy terminus. The encoded protein functions as a ribonuclease and is required by the RNA interference and small temporal RNA (stRNA) pathways to produce the active small RNA component that represses gene expression.

Transmembrane protease serine 3 (TMPRSS3): This gene encodes a protein that belongs to the serine protease family. The encoded protein contains a serine protease domain, a transmembrane domain, a LDL receptor-like domain, and a scavenger receptor cysteine-rich domain. Serine proteases are known to be involved in a variety of biological processes, whose malfunction often leads to human diseases and disorders. This gene was identified by its association with both congenital and childhood onset autosomal recessive deafness. This gene is also identified as a tumor associated gene and is overexpressed in ovarian tumors.

Histone cluster 1 H2BO (HIST1H2BO): Histones are basic nuclear proteins that are responsible for the nucleosome structure of the chromosomal fiber in eukaryotes. Two molecules of each of the four core histones (H2A, H2B, H3, and H4) form an octamer, around which approximately 146 by of DNA is wrapped in repeating units, called nucleosomes. The linker histone, H1, interacts with linker DNA between nucleosomes and functions in the compaction of chromatin into higher order structures. This gene is intronless and encodes a member of the histone H2B family. Transcripts from this gene lack polyA tails but instead contain a palindromic termination element. This gene is found in the small histone gene cluster on chromosome 6p22-p21.3.

BCL2-associated X protein (BAX): Overexpression of BAX sensitizes human pancreatic cancer cells to apoptosis induced by chemotherapeutic agents. Enhanced BAX expression may have therapeutic application in enhancing the efficacy of chemotherapy in pancreatic cancers. The current combination of the anti-cancer compounds Gemcitabine and P276-00 as provided in the instant disclosure reveals a significant up regulation of BAX upon exposure.

Cytochrome C: Cytochrome C is an intermediate in apoptosis, a controlled form of cell death used to kill cells in the process of development or in response to DNA damage. A variety of apoptotic stimuli cause Cytochrome C release from mitochondria, which in turn induces a series of biochemical reactions that result in caspase 3 activation and subsequent cell death. The data on the combination of Gemcitabine and P276-00 reveals release of Cytochrome C from mitochondria.

Caspase-3: Caspases are crucial mediators of programmed cell death (apoptosis). Among them, caspase-3 is a frequently activated death protease, catalyzing the specific cleavage of many key cellular proteins like PARP. The data provided in the instant disclosure reveals activation of cleaved caspase-3 upon treatment with the combination of Gemcitabine and P276-00.

Phospho RB (pRB): Overexpression of pRB is associated with human pancreatic duct-cell cancer and may allow pancreatic cancer cells to evade chemotherapy-induced apoptosis. The present studies revealed that pRB expression was down regulated by the combination of Gemcitabine and P276-00.

Phospho AKT (pAKT): The role of AKT in carcinogenesis has been well documented and AKT is overexpressed in a variety of human cancer types. AKT has been associated with the initiation of tumorigenesis in pancreatic cancer and gliomas and seems to correlate with stage and tumor grade in prostate cancer. AKT activation is correlated with higher histologic tumour grade (P=0.047). Thus, it is suggested that AKT is frequently activated in pancreatic cancer. Further its antiapoptotic activity may be mediated by HER-2/neu overexpression. In the current studies disclosed herein, pRB expression was down regulated by the combination of Gemcitabine and P276-00.

Cell division cycle 25 homolog B (CDC25B): CDC25B inhibitors reduce the growth of pancreatic cancer cell lines, resulting in the accumulation of phosphorylated CDC2 and G2/M arrest. These findings raise the possibility that inhibition of CDC25B phosphatase may ultimately have a therapeutic role in this disorder. Current data reveals inhibition of CDC25B expression upon treatment with the combination of Gemcitabine and P276-00.

CyclinD1: Inhibition of Cyclin D1 expression in human pancreatic cancer cells is associated with increased chemosensitivity and decreased expression of multiple chemoresistance genes like MDR-1 and P-glycoprotein. The current studies also reveal downregulation of cyclinD1 expression in combination of Gemcitabine and P276-00.

Pleiotrophin (PTN): PTN is overexpressed in a variety of neuroectodermal tumors and described as an essential angiogenic growth factor in choriocarcinoma and melanoma, promoting metastatic growth. PTN is an essential growth factor for pancreatic cancer. Due to the restricted expression pattern of PTN in adults, PTN is suggested as a target for pancreatic cancer therapy. The current data reveals inhibition of PTN expression on treatment with the combination of Gemcitabine and P276-00.

Matrix Metalloproteinase-1 (MMP-1): The involvement of MMPs in various malignancies, including pancreatic cancer, makes them attractive as potential pharmacological or genetic targets for antitumor therapies. Administration of the combination of Gemcitabine and P276-00 disclosed herein, reveals the down regulation of MMP-1.

Vascular endothelial growth factor (VEGF): VEGF is a potent angiogenic factor that also has the ability to increase vascular permeability. VEGF plays an important role in the development of malignant ascites in various cancers. The data of the combination of Gemcitabine and P276-00 as presented in the current disclosure reveals the down regulation of VEGF.

B-cell CLL/lymphoma 2 (BCL-2): Increased BCL2 expression correlates with apoptotic resistance and metastatic potential in different type of tumors. Bcl-2-specific siRNAs restore Gemcitabine sensitivity in human pancreatic cancer cells. The current data reveals down regulation of bc-2 expression upon administration of the combination of Gemcitabine and P276-00..

Drug response in patients administered with the combination of Gemcitabine and P1446A is monitored using a gene signature comprising at least two markers selected from the group consisting of P21, REV3L, FGF5, PTK7, POLH, P27, and SSTR2.

Expression of these markers is up regulated upon administration of the combination of Gemcitabine and P1446A.

These gene markers are herein further described:

Cyclin-dependent kinase inhibitor 1A (P21): This gene encodes a potent cyclin-dependent kinase inhibitor. The encoded protein binds to and inhibits the activity of cyclin-CDK2 or -CDK4 complexes, and thus functions as a regulator of cell cycle progression at G1 phase of the cell cycle. The expression of this gene is tightly controlled by the tumor suppressor protein p53, through which this protein mediates the p53-dependent cell cycle G1 phase arrest in response to a variety of stress stimuli. This protein can interact with proliferating cell nuclear antigen (PCNA), a DNA polymerase accessory factor, and plays a regulatory role in S phase DNA replication and DNA damage repair.

REV3-like, catalytic subunit of DNA polymerase zeta (REV3L): Cell cycle checkpoints and DNA repair act in concert to ensure DNA integrity during perturbation of normal replication or in response to genotoxic agents. Deficiencies in these protective mechanisms can lead to cellular transformation and ultimately tumorigenesis. REV3, the catalytic subunit of the low-fidelity DNA repair polymerase zeta and plays a role in double-strand break (DSB)-induced DNA repair by homologous recombination and reduced expression of REV3 is independent of the carcinoma stages, suggesting that the downregulation of REV3 might have occurred early during tumorigenesis.

Protein Tyrosine Kinase 7 (PTK7): Receptor protein tyrosine kinases transduce extracellular signals across the cell membrane. A subgroup of these kinases lack detectable catalytic tyrosine kinase activity but retain roles in signal transduction. The protein encoded by this gene is a member of this subgroup of tyrosine kinases and may function as a cell adhesion molecule. This gene is thought to be expressed in colon carcinomas but not in normal colon, and therefore may be a marker for or may be involved in tumor progression.

Cyclin-dependent kinase inhibitor 1B (P27): This gene encodes a cyclin-dependent kinase inhibitor, which shares a limited similarity with CDK inhibitor CDKN1A/p21. The encoded protein binds to and prevents the activation of cyclin E-CDK2 or cyclin D-CDK4 complexes, and thus controls the cell cycle progression at Gl. The degradation of this protein, which is triggered by its CDK dependent phosphorylation and subsequent ubiquitination by SCF complexes, is required for the cellular transition from quiescence to the proliferative state.

Somatostatin Receptor 2 (SSTR2): Somatostatin acts at multiple sites to inhibit the release of many hormones and other secretory proteins. The biologic effects of somatostatin are probably mediated by a family of G protein-coupled receptors that are expressed in a tissue-specific manner. SSTR2 is a member of the superfamily of receptors having seven transmembrane segments and is expressed in highest levels in cerebrum and kidney. Introduction of the SSTR2 gene, the expression of which is frequently lost in human pancreatic adenocarcinoma, exerts anti-angiogenic effects by down regulating the expression of the factors, which are involved in tumor angiogenesis and metastasis, suggesting SSTR2 gene transfer as a promising strategy for gene therapy for pancreatic cancer.

In yet another embodiment, the invention provides a method of monitoring the drug response in a patient administered with the combination of Gemcitabine and P276-00. The method involves detection of the gene signature wherein the gene markers SNX7, FA38A, DNAI1, BAX, Cytochrome C and Caspase-3 are up regulated and RRM2, CDK8, DLG5, FGF5, MKKS, HELLS, PPIL4, SLC19A2, ID1, DICER1, TMPRSS3, HIST1H2BO, pAKT, pRB, CyclinD1, MMP-1, VEGF, CDC25B, P21, P14ARF and PTN are down regulated.

In a further embodiment, a method of monitoring the drug response in a patient administered with the combination of Gemcitabine and P1446A is provided. The method involves detection of the gene signature wherein up regulation of the gene markers P21, REV3L, FGF5, PTK7, POLH, P27, and SSTR2 is observed.

In the context of the present invention, the terms “subject” and “patient” are used interchangeably which generally refers to an individual (preferably human) who is suffering from cancer and is in need of treatment for cancer. Moreover, said terms also connote to the term “cancer patient” as used herein and in the appended claims.

Further, in the context of the present invention, the terms “drug response markers”, “gene markers”, “markers” or “cancer markers” are used interchangeably throughout the specification.

The following examples provide illustrative embodiments of the invention. A person skilled in the art will readily recognize the various modifications and variations that may be performed without altering the scope of the present invention. Such modifications and variations are encompassed within the scope of the invention and the examples do not in any way limit the scope of the invention.

### EXAMPLES

### Example 1

### Propidium Iodide-Based Fluorescence Cytotoxicity Assay

The combination of P276-00 and Gemcitabine was screened for its synergistic effect in Panc-1 cells using propidium iodide based fluorescence cytotoxicity assay.

Test System: The Panc-1 cell line was procured from ATCC (American Tissue type Culture Collection), USA. Catalog number: CRL-1469 and the frozen vial was stored in liquid nitrogen container. Propidium iodide dye was procured from Sigma-Aldrich, USA. Catalog number: P-4170-100 mg and it was stored at 2-8° C.

Method: Panc-1 cells were seeded at a density of 2000 cells/well, in a 200 μL in tissue culture grade 96 well plate and allowed to recover for 24 hrs in a humidified 5%±0.2 CO2 incubator at 37° C.±0.5° C. After 24 hrs, 1 μL of 200 X (200 times higher than required concentration is denoted as 200×) compound (dissolved in neat DMSO), as per Table 1 or 2 was added to the wells. The final DMSO concentration was 0.5% in wells. The plate was incubated for 24 hrs in humidified 5%±0.2 CO₂ incubator at 37±0.5° C.

After 24 hrs Minimum Essential Medium (MEM) from the Gemcitabine treated wells was removed and washed two times with fresh MEM and followed by addition of 200 μL of fresh MEM per well. 1 μL of 200× (200 times higher than required concentration is denoted as 200×) compound (dissolved in neat DMSO) was then added as per the Table 1 and 2 designs. The final DMSO concentration was 0.5% in wells which also served as the vehicle control for the study. After 72 hrs the plate was removed from CO₂ incubator and spent MEM aspirated from the wells and supplemented with fresh MEM. This was followed by addition of 25 μL of propidium iodide (50 μg/ml in medium) per well. The same plate was frozen at −80° C. for 24 hrs and then thawed and allowed to come to room temperature. The fluorescence at a wavelength of 530 nM excitation and 620 nM emission was read.

The percent cytotoxicity was calculated using the following formula

$$Percent Cytotoxicity = \frac{( Reading of Control - Reading of Treated cells ]}{Reading of Control} \times 100$$

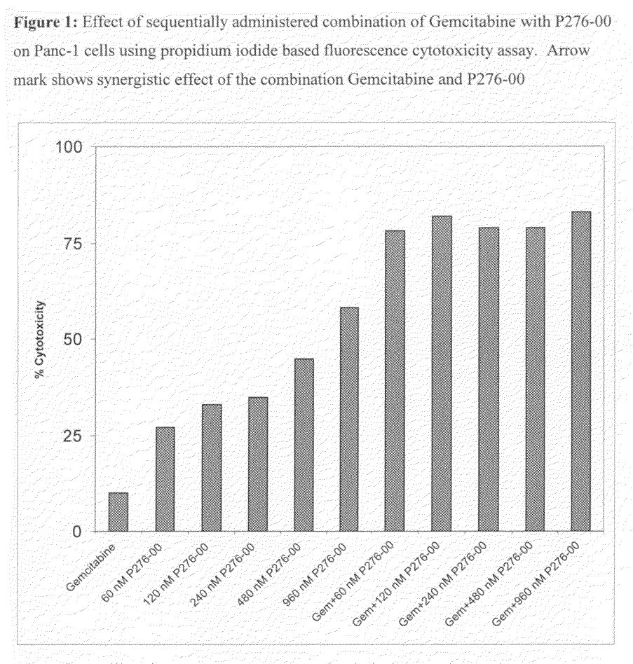

Results: The propidium iodide based cytotoxicity assay showed that Gemcitabine (0.1 nM) IC₁₀ for 24 hrs followed by P276-00 (60 nM) IC₁₀ for 72 hrs showed synergistic toxicity of 78% (FIG. 1, Table 2). The results of in vitro testing showed that these two agents when tested alone produce minimal cytotoxicity at IC₁₀ (Table 1) but when tested in combination at IC₁₀, the two agents exhibited additional antiproliferative/cytotoxic effects across a range of dosages (Table 2, FIG. 1). The synergistic effect of gemcitabine and P276-00 combination not only make cancer cells more susceptible but further facilitate use of lower effective drug doses in patients.

TABLE 1
Effect of administration of Gemcitabine or P276-00 on Panc-1 cells

| Concentration (nM) | Fluorescence Unit | % Cytotoxicity |
|---|---|---|
| Gemcitabine 0.1 nM | 31455 | 10 |
| 60 nM P276-00 | 24558 | 27 |
| 120 nM P276-00 | 22768 | 33 |
| 240 nM P276-00 | 22212 | 35 |
| 480 nM P276-00 | 18831 | 45 |
| 960 nM P276-00 | 15416 | 58 |
| Control | 34599 | 0 |

TABLE 2
Effect of sequentially administered combination
of Gemcitabine (Gem) with P276-00 on Panc1 cells

| | Fluorescence | |
|---|---|---|
| Gem. at 0.1 nM for 24 hrs + P276-00 | unit | % Cytotoxicity |
| 60 nM P276-00 for 72 hrs | 12147 | 78 |
| 120 nM P276-00 for 72 hrs | 10384 | 82 |
| 240 nM P276-00 for 72 hrs | 11851 | 79 |
| 480 nM P276-00 for 72 hrs | 11796 | 79 |
| 960 nM P276-00 for 72 hrs | 9654 | 83 |
| Control | 55847 | 0 |

### EXAMPLE 2

### Cell Cycle Analysis by DNA Content

Understanding the status of cells cycles G1, S, G2 and M is indicative of the cytotoxic effect including apoptosis and DNA damage of anticancer drugs. In the current study FACS was used to measure the cell cycle stages in Panc 1 cells after exposure to anticancer drugs including drug combination. The synergistic effect of Gemcitabine with P276-00 on Panc-1 cancer cells was analyzed using propidium iodide based cell cycle analysis where total population of cells was sorted as sub G0, G1, S and G2/M populations according to total fluorescent intensity.

Dosage

P276-00 at a final concentration of 60 nM, 240 nM and 480 nM and Gemcitabine at a final concentration of 1 nM, 3 nM and 10 nM were analyzed in single dose and in all possible combinations of the dose range for the Gemcitabine and P276-00.

Methodology: Cell Cycle Analysis Using Flow Cytometry

Panc-1 cells were treated with Gemcitabine for 0 to 24 hrs. After 24 hrs the cells were washed twice with plain MEM. Fresh MEM with 10% serum (2 mL/well) was added to the wells, followed by treatment with P276-00 for 24 hrs to 96 hrs. After 96 hrs the treated cells were harvested, fixed, stained with propidium iodide and analyzed for specific cell arrest using DNA content analysis by flow cytometry.

Adherent cells were trypsinized and washed with PBS or HBSS without Ca++ and Mg++ containing EDTA pH 8.0 and BSA. They were further isolated, centrifuged and total number of cells were counted and recorded. The pellet of cells was resuspended in ice-cold PBS and a uniform suspension was prepared. Cold ethanol was slowly added to the suspension while vortexing it. The cells were then fixed at 4° C. overnight.

The cells were then taken in a conical tube and centrifuged to remove the ethanol. The pellet was resuspended in PBS and calf serum after vortexing and washing it in the same. Propidium iodide and boiled RNase S was added to it and incubated at 37° C. for 30 mins. Analysis was carried out using a flow cytometer (Becton Dickinson FACSCalibur, a 4-color, dual-laser benchtop flow cytometer).

Results

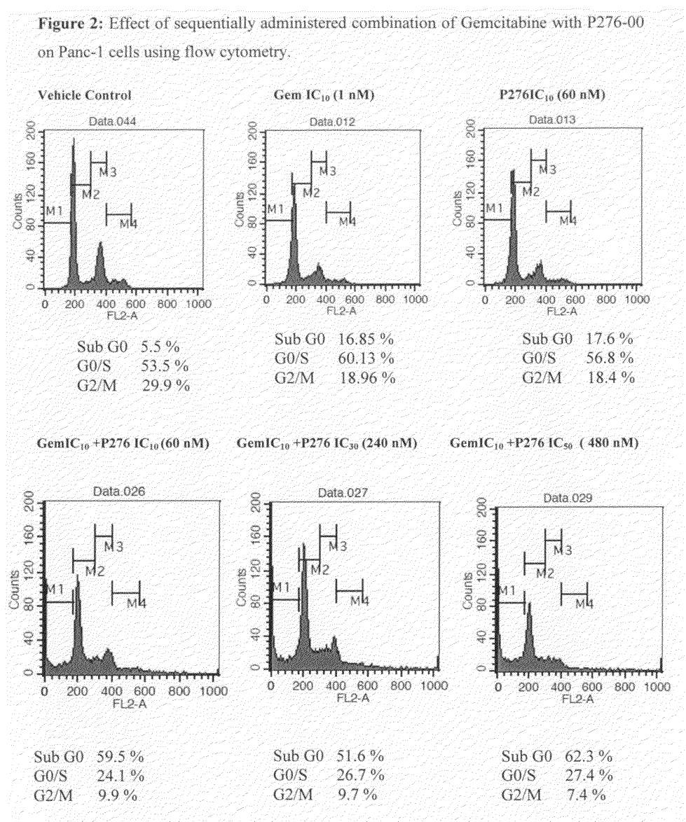

Cancer cells were exposed to IC₁₀ and IC₅₀ values of drugs alone and in combination. After that cells are fixed and stained with propidium iodide to analyze the cell cycle checkpoints. Cells exposed to P276-00, gemcitabine, and their combinations presented typical apoptotic morphology with cell shrinkage, nuclear condensation and fragmentation, and rupture of cells into debris. The results of the cell cycle analysis revealed that control cells showed 5.5% population of cells in sub G0 (apoptotic cells) (FIG. 2). Gemcitabine 1 nM treated cells showed 16.85% population of cells in sub G0 and P276-00 60 nM treated cells showed 17.6% population of cells in sub G0. Interestingly, when cells were exposed to the combination of Gemcitabine (1 nM) for 24 hr followed by P276-00 (60 nM, 240 nM and 480 nM) for 72 hr, they showed the highest apoptotic index as shown by the sub G0 population of 59.5%, 51.6% and 61.3% respectively. These results were confirmed by the enhancement of the sub-G1 region on the DNA content histograms demonstrating that, after drug treatments, cell-cycle modulation was accompanied by the induction of apoptosis.

Furthermore it was observed that in combination of gemcitabine and P276-00, there was a 50% reduction in both G0-S and G2-M population, which could be due to the fact that cells that were initially arrested in both G0-S and G2-M later entered into apoptosis leading to the sharp increase in Sub G0 population. These results further substantiate the synergistic activity displayed by Gemcitabine with P276-00.

### EXAMPLE 3

### Microarray Analysis to Detect Gene Expression Signatures Associated with the in Vitro Synergistic Effect of the Combination Therapy of Gemcitabine with P276-00 in Panc-1 Cells

The goal of the microarray analysis was to evaluate putative gene expression signatures associated with the in vitro synergistic effect of the combination therapy of Gemcitabine with P276-00 in Panc-1 cells.

The time points (3 hrs, 6 hrs, 12 hrs) were chosen to establish early molecular changes in the transcription profile for probable biomarkers and mechanism of action for the combination therapy.

Methodology: Sample Processing and RNA Extraction

Approximately 20 million cells were lysed in Trizol Reagent (Invitrogen Corp, USA) and passed through a 21-gauge needle for 5-10 times before RNA extraction was performed. Total RNA extraction was performed using the RNeasy Mini kit (Qiagen, USA) according to the manufacturer's protocol. The final total RNA was eluted in nuclease-free water and the concentration/purity was determined by Nanodrop spectrophotometer (Thermo Fisher Scientific, Delaware, USA).

cDNA generation and labeling: 20 μg of total RNA was reverse transcribed using amino allyl dUTP and oligo dT, the resulting cDNA was indirectly labeled with Cy3/Cy5 dyes (GE Healthcare, USA) using a fluorescent labeling kit (Promega Biosciences, USA). The concentration and dye incorporation of the labeled products were determined by Nanodrop spectrophotometer (Thermo Fisher Scientific, Delaware, USA). Equal amounts of labeled cDNA were denatured at 95° C. for 5 mins, cooled and microfuged at 13200 rpm for 5 mins before hybridization.

Array fabrication and hybridization: 70-mer transcript oligonucleotides representing 36,480 human genes was purchased from Operon Biotechnologies (Germany) and spotted onto aminosilane coated glass slides (ArrayIt, USA) by contact printing using a Omnigrid (OG100) spotter (Genomic Solutions Inc, USA). Post printing, the slides were cross-linked with UV light at 650 mJ/sec2, vacuumed, sealed and stored in the dark.

The slides were pre-processed for background reduction using Pronto (Promega Life sciences, USA) pre-hyb kit as per the manufacturer's instructions. Hybridization was carried out in the GeneTAC Hybstation (Genomic Solutions Inc, USA) for 18 hrs. The hybridization protocol is as follows: 6 hrs at 42° C. (with agitation), 6 hrs at 35° C. (with agitation), and 6 hrs at 30° C. (with agitation). Post hybridization, the arrays were washed according to the manufacturer's protocol. The slides were dried by centrifugation at 1600 rpm for 5 mins.

Scanning and image analysis: Post drying, the slides were scanned in a laser scanner (Genomic Solutions, USA) for Cy3 and Cy5 detection. The PMT settings were adjusted using the auto-exposure feature of the scanner. The TIFF files thus generated were exported to GeneTAC Integrator software (Genomic Solutions, USA) for image analysis. The resultant CSV file containing the annotated genome and the corresponding ratios were further used for data analysis.

Results: The combination of Gemcitabine and P276-00 used for the treatment in Panc-1 cell line induces a dynamic gene expression change with respect to compound action, time of dosing and the concentrations. The major gene families that have been dys- regulated by combination treatment are the proteins of the serine-threonine kinase family (Table 3). The IC₅₀ doses also up regulate the inflammatory pathways downstream of TNFR family without the induction of NFKB1 indicating the inhibition of the pro-survival pathways in Panc-1 cell line.

TABLE 3
Gene expression values following drug exposure in Panc-1 cell lines

| Gene ID | Gene Name | Gemcitabine | P276 | Combination |
|---|---|---|---|---|
| HIST1H2BO | Histone 1, H2BO | −0.71 | −0.39 | −3.28 |
| TMPRSS3 | Transmembrane protease, serine 3 | 0.85 | 0.33 | −1.92 |
| DICER1 | Dicer1, Dcr-1 homolog | −0.16 | 0.14 | −1.74 |
| ID1 | Inhibitor of DNA binding 1 | −0.60 | 0.18 | −1.24 |
| SLC19A2 | Solute carrier family 19 member 2 | 0.37 | 0.97 | −1.02 |
| PPIL4 | Peptidylprolyl isomerase -like 4 | 0.36 | 0.30 | −1.04 |
| HELLS | Helicase, lymphoid-specific | 0.40 | 0.62 | −1.05 |
| MKKS | McKusick-Kaufman syndrome | −0.02 | −0.08 | −1.86 |
| FGF5 | Fibroblast growth factor 5 | 0.28 | 0.23 | −0.49 |
| DLG5 | Discs, large homolog 5 | −0.19 | 0.25 | −0.46 |
| CDK8 | Cyclin-dependent kinase 8 | −0.03 | 0.16 | −0.61 |
| RRM2 | Ribonucleotide reductase M2 | 0.69 | −0.14 | −0.94 |
| DNAI1 | Dynein, | 0.03 | −0.20 | 2.02 |
| FA38A | FA38A | −0.70 | −0.93 | 1.96 |
| SNX7 | Sorting nexin 7 | 0.27 | −0.12 | 1.93 |

*Numerical value indicates log value of intensities as obtained by microarray experiments. Values close to zero indicate normal expression, negative value denotes down regulation and positive value indicates up-regulation of gene expression.

### EXAMPLE 4

### CCK-8 Based Cytotoxicity Assay

The combination of Gemcitabine with P1446A was screened for its synergistic effect in Panc-1 by CCK-8 (Cell counting kit-8) based cytotoxicity assay. CCK-8 based cytotoxicity assay is a sensitive nonradioactive colorimetric assay for determining the number of viable cells in cell proliferation and cytotoxicity assays. WST-8 (2-(2-methoxy-4-nitrophenyl)-3-(4-nitrophenyl)-5-(2,4-disulfophenyl)-2H tetrazolium, monosodium salt) is bioreduced by cellular dehydrogenases to an orange formazan product that is soluble in tissue culture medium. The amount of formazan produced is directly proportional to the number of living cells.

Test System: The Panc-1 cell line was procured from ATCC (American Tissue type Culture Collection USA, catalog number: CRL-1469) and the frozen vial was stored in liquid nitrogen container.

Methodology: Gemcitabine at final concentrations of 1 nM, 10 nM and 100 nM and P1446A at a final concentration of 3 nM, 10 nM, 30 nM, 100 nM and 300 nM were analyzed in single dose and in all possible combinations of the dose range for the two drugs mentioned above. Panc-1 cells were seeded at a density of 3000 cells/well in a 96 well plate and allowed to recover for 24 hrs in a CO₂ incubator at 37° C. Subsequently, compounds (Gemcitabine and/or P1446A) were added to the plates. The final DMSO concentration was 0.5% in wells. Plates were incubated for 24, 48, and 72 hrs. At the end of incubation CCK-8 was added and kept at 37° C. for 3 hrs following which absorbance was measured at 450 nM.

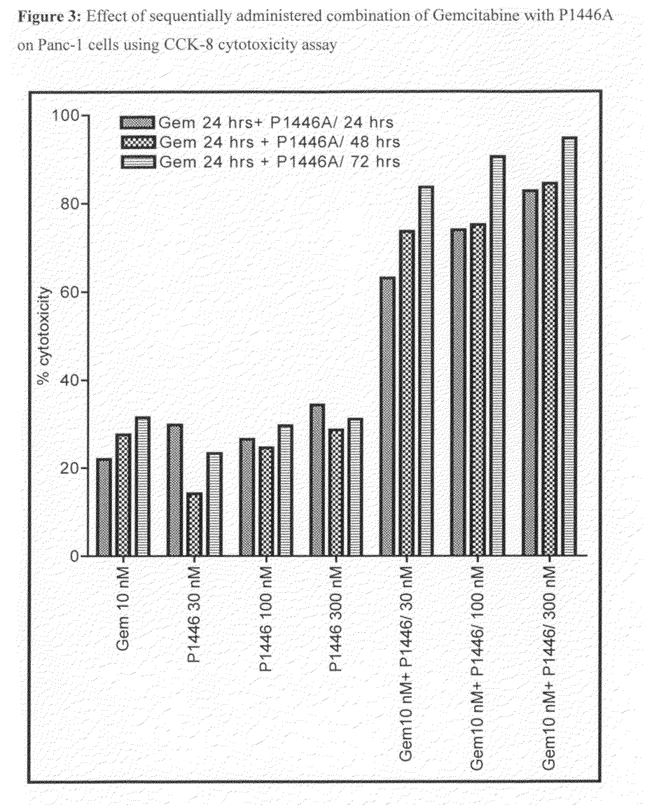

Results: Combination of Gemcitabine and P1446A was observed to exhibit synergistic effect at a concentration of 10 nM and 100 nM respectively. Gemcitabine at 10 nM showed cytotoxicity of 31.47% while P1446A at 100 nM, showed cytotoxicity of 29.6%. However when used as a combination of Gemcitabine 10 nM for 24 hrs, followed by incubation with P1446A 100 nM for 72 hrs, an increase in cytotoxicity to 90.5% was noted, which is at least 29% more cytotoxicity than the additive effect of the two agents suggesting a marked synergistic effect of the combination (Table 4). Gemcitabine and P1446A were also found to be more synergistic at the 10 nM and 300 nM respectively. Gemcitabine at 10 nM showed cytotoxicity of 31.47% and P1446A at 300 nM, showed cytotoxicity of 31.05%. However when used as a combination of Gemcitabine 10 nM for 24 hrs, followed by P1446A 300 nM for 72 hrs an increase in cytotoxicity to the extent of about 95% was noted, which is 33% more cytotoxicity than the additive effect suggesting a significant synergy of the combination drug (Table 4, FIG. 3).

TABLE 4
Effect of combination of Gemcitabine with P1446A in Panc-1 cells

% Cytotoxicity

| Concentration in nM | 24 hrs | 48 hrs | 72 hrs |
|---|---|---|---|
| Gem 10 nM | 21.997 | 27.571 | 31.471 |
| P1446A 30 nM | 29.825 | 14.163 | 23.33 |
| P1446A 100 nM | 26.525 | 24.596 | 29.603 |
| P1446A 300 nM | 34.36 | 28.636 | 31.075 |
| Gem10 nM + P1446A/30 nM | 63.099 | 73.640 | 83.655 |
| Gem10 nM + P1446A/100 nM | 73.935 | 75.136 | 90.527 |
| Gem10 nM + P1446A/300 nM | 82.788 | 84.482 | 94.720 |

Gem = Gemcitabine

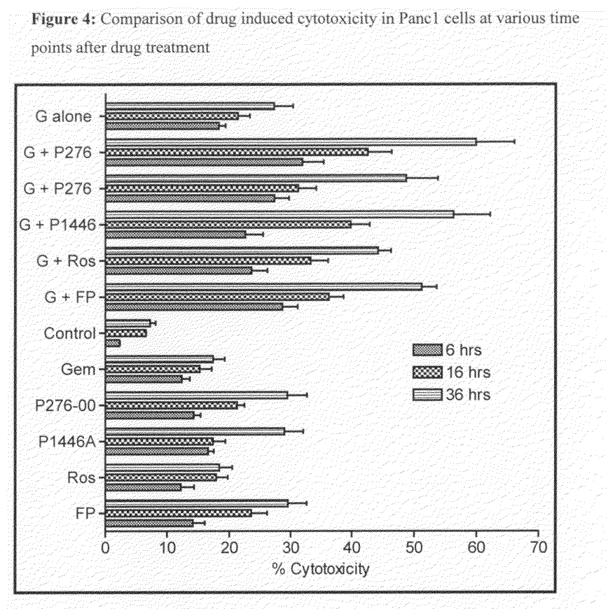

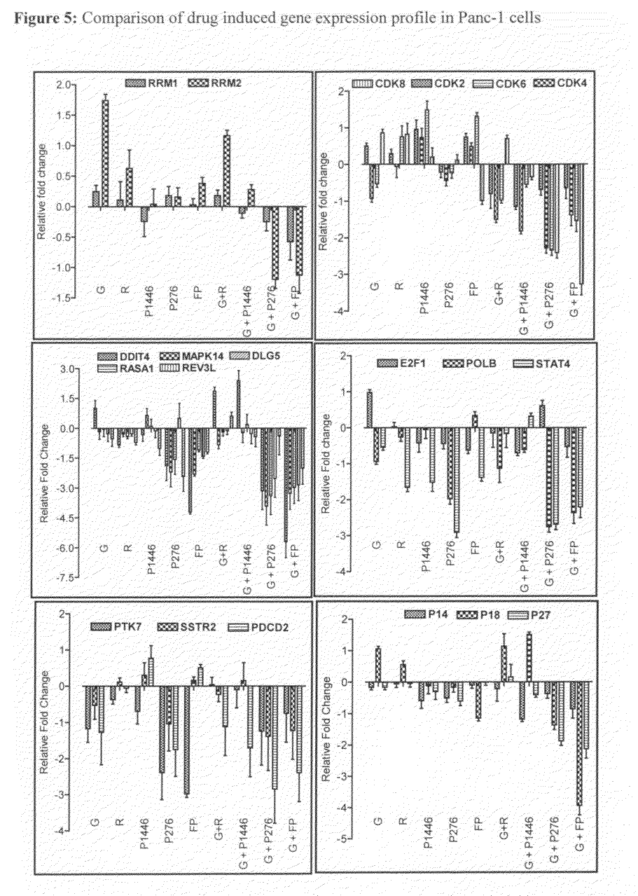

Subsequently cytotoxic effect of Gemcitabine, P276-00 and P1446A alone and in combinations was compared with known CDK inhibitors such as Flavopiridol (FP) and Roscovitine (R) at various time points such as 6, 16 and 36 hrs. It was observed that P276-00 when combined with Gemcitabine showed cytotoxicity of 22.54%, 39.78%, 56.32% at time points 6, 16, 36 hrs post drug treatment respectively which is significantly higher or comparable to combination of flavopiridol and gemcitabine. as compared to other CDK inhibitor combinations used in the current study (Table 5, FIG. 4).

Data from Table 5 also indicates that P1446A is relatively less potent than flavopiridol or P276-00 as shown by cytotoxicity data. However, unlike all the other drugs from the Table 5, P1446A is an oral drug and hence highly desirable by the clinics and the patients.

TABLE 5
Drug induced cytotoxicity in Panc1 cells at
various time points after drug treatment

| | % Cytotoxicity in Panc1 cells | | |
|---|---|---|---|
| Drugs | 6 hrs | 16 hrs | 36 hrs |
| Gemcitabine | 2.31 | 6.54 | 7.23 |
| Roscovitine | 12.35 | 15.24 | 17.35 |
| P1446A | 12.23 | 17.87 | 18.37 |
| P276-00 | 16.58 | 17.35 | 28.98 |
| Flavopiridol | 14.25 | 21.25 | 29.47 |
| Gemcitabine + Roscovitine | 14.15 | 23.54 | 29.56 |
| Gemcitabine + P1446A | 23.56 | 33.24 | 44.21 |
| Gemcitabine + P276-00 | 22.54 | 39.78 | 56.32 |
| Gemcitabine + Flavopiridol | 28.65 | 36.25 | 51.24 |

### EXAMPLE 5

### Gene expression profile using RTQ-PCR

The objective of the experiment was to evaluate the synergistic effect of drug combination (Gemcitabine with P1446A) as compared to individual use of the same. The synergistic effect of the compound or drug combination in the Panc-1 cell line was measured in terms of gene expression and expressed as fold changes as compared to the cell control with no drug treatment.

Methodology: Cell lines treated with drugs (Gemcitabine, P1446A or both) were used for total RNA isolation using a commercial RNA extraction kit (Qiagen Corporation, Germany). The first-strand cDNA was synthesized from total RNA using first strand cDNA synthesis kit from Invitrogen Corporation (California, USA). This was followed by real time quantitative polymerase chain reaction (RTQ PCR) using gene specific primers and standard thermal program of initial denaturation at 95° C. for 5 mins and 40 cycles of 95° C. for 10 seconds, followed by 6° C. for 30 seconds (Realplex PCR machine from Eppendorf, Germany). Quantitative measurement of products made during PCR cycles was normalized against a housekeeping gene (Actin) and used to measure the gene expression as fold changes as compared to respective control (FIG. 5).

Results: Gene markers, which showed up regulation in response to combination therapy of Gemcitabine with P1446A, are listed in Table 6.

TABLE 6
Gene expression profile for cancer markers in Panc1 cell line after
exposure to Gemcitabine, P1446A and combination of both
Gene expression in log 2 fold ratio as compared to control cells with no drug treatment

| | E2F1 | POLB | STAT4 | SSTR2 | PTK7 | PDCD2 | MAPK1 | RASA1 | REV3L | DLG5 | PTN |
|---|---|---|---|---|---|---|---|---|---|---|---|
| 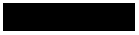 | 0.98 | −0.94 | −0.54 | −1.17 | −0.53 | −1.27 | −0.18 | −0.04 | −0.28 | −0.53 | 0.68 |
| 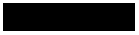 | 0.03 | −0.26 | −1.66 | −0.37 | 0.11 | −0.05 | −0.29 | −0.41 | −0.27 | −0.71 | −0.28 |
| 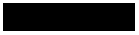 | −0.43 | −0.05 | −1.52 | −0.69 | 0.30 | 0.77 | 0.64 | 0.10 | −0.13 | −1.01 | 0.40 |
| 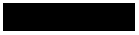 | −0.44 | −1.98 | −2.91 | −2.39 | −1.04 | −1.75 | −2.20 | −1.57 | 0.50 | −2.43 | 0.06 |
| 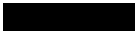 | −0.62 | 0.34 | −1.39 | −2.98 | 0.16 | 0.50 | −2.31 | −1.10 | −1.42 | −1.19 | −0.24 |
| 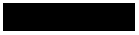 | −0.15 | −1.13 | −0.16 | 0.04 | −0.23 | −1.12 | −0.83 | −0.19 | −0.13 | 0.60 | −0.22 |
| 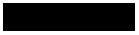 | −0.70 | −0.61 | 0.32 | −0.10 | 0.15 | −1.71 | −0.22 | 0.18 | −0.28 | −0.43 | −0.07 |
| 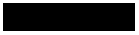 | 0.61 | −2.76 | −2.69 | −1.24 | −1.39 | −2.85 | −3.92 | −3.40 | −2.53 | −0.39 | −1.23 |
| 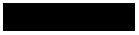 | −0.53 | −2.37 | −2.21 | −0.75 | −1.23 | −2.40 | −3.28 | −2.99 | −2.85 | −2.02 | −0.35 |

| | CDK2 | CDK4 | CDK6 | CDK8 | p14 | p18 | p27 | RRM1 | RRM2 | DDIT4 |
|---|---|---|---|---|---|---|---|---|---|---|
| 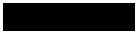 | 0.50 | −0.93 | −0.53 | 0.86 | −0.17 | 1.06 | −0.16 | 0.25 | 1.74 | 1.01 |
| 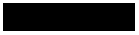 | 0.29 | −0.06 | 0.75 | 0.82 | −0.05 | 0.56 | −0.04 | 0.11 | 0.63 | −0.84 |
| 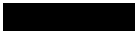 | 0.96 | 0.73 | 1.48 | 0.20 | −0.59 | −0.12 | −0.30 | −0.24 | 0.04 | −0.31 |
| 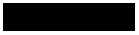 | −0.21 | −0.44 | −0.23 | 0.11 | −0.50 | −0.17 | −0.60 | 0.18 | 0.16 | −1.88 |
| 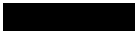 | 0.74 | 0.49 | 1.31 | −0.99 | −0.10 | −1.14 | −0.01 | 0.03 | 0.38 | −4.21 |
| 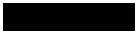 | −0.80 | −1.50 | −0.97 | 0.70 | −0.21 | 1.14 | 0.16 | 0.18 | 1.16 | 1.86 |
| 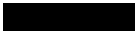 | −1.15 | −1.82 | −0.54 | −0.34 | −1.18 | 1.51 | −0.41 | −0.11 | 0.28 | 2.39 |
| 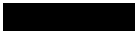 | −0.69 | −2.28 | −2.34 | −2.41 | −0.37 | −1.37 | −1.88 | −0.25 | −1.20 | −3.15 |
| 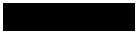 | −0.64 | −1.38 | −1.54 | −3.27 | −0.86 | −3.95 | −2.13 | −0.58 | −1.13 | −5.71 |

G = Gemcitabine, R = Roscovitine, FP = Flavopiridol

### EXAMPLE 6

### Protein Expression Studies Using High Content Cell Imaging System

In the current experiment, the synergy of drug combination at protein level was validated to support the gene expression data since proteins are directly involved in the biological processes. The data was generated using cell imaging system, where, cells after drug exposure were treated with protein specific antibody, followed by detection markers for the antibody. The images were generated and protein levels quantitated as an indication of drug effect.

Methodology: Panc-1 cells were seeded at a density of 5000 cells per well in 96 well plates and allowed recovery for 24 hrs in 5% CO₂ incubator. After recovery, cells were challenged with Gemcitabine (IC₃₀) for 24 hrs. After 24 hrs Gemcitabine was removed and fresh medium was added. The second drug was added at a concentration of IC₉₀ for a period of 6 hrs, 16 hrs and 36 hrs. The combination efficacy was identified against single treatments. At the end of the experiment Panc-1 cells were fixed and permeabilized. All the primary antibodies were added at a concentration according to manufacture protocol. After one hour incubation the cells were washed three times and labeled with secondary antibodies tagged with Dye Light 548 and nucleus was stained with Hoechest3342. After antibody labeling, cells were stained with the Cellomics whole Cell Stain Green to identify the cell's cytoplasmic area. Then all the plates were scanned in Cellomics ArrayScan® VTI HCS Reader. The results were analyzed using software that is designed for measuring the read from the imaging system.

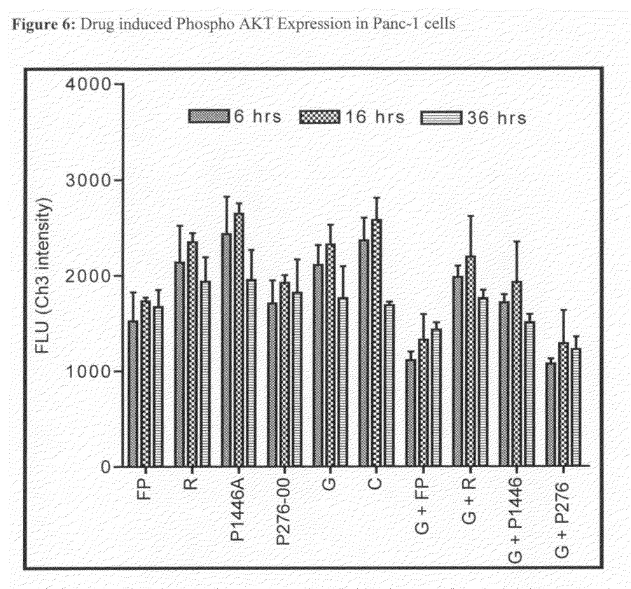

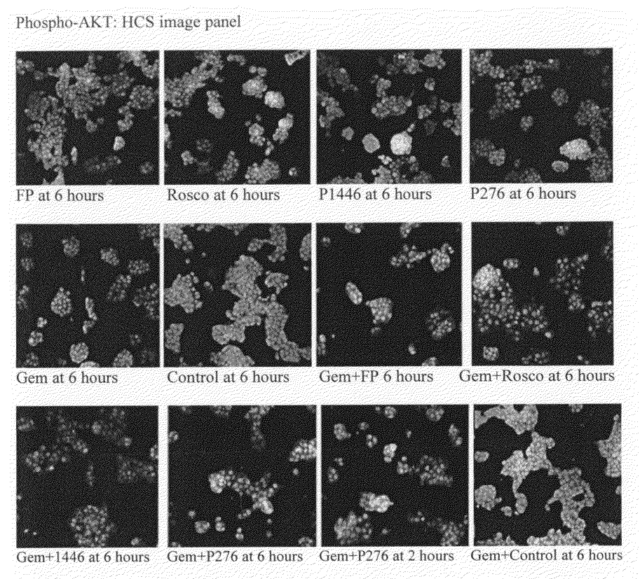

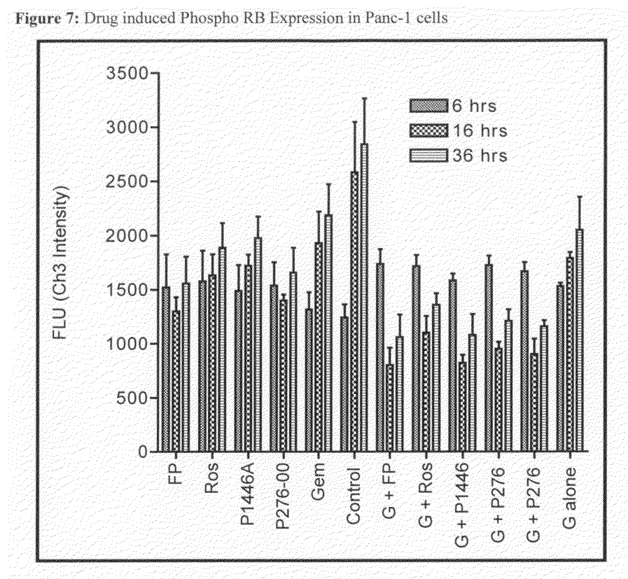

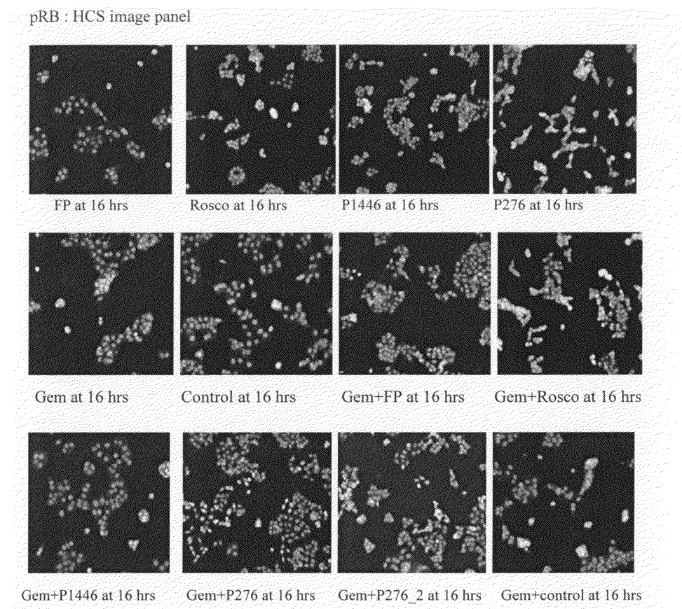

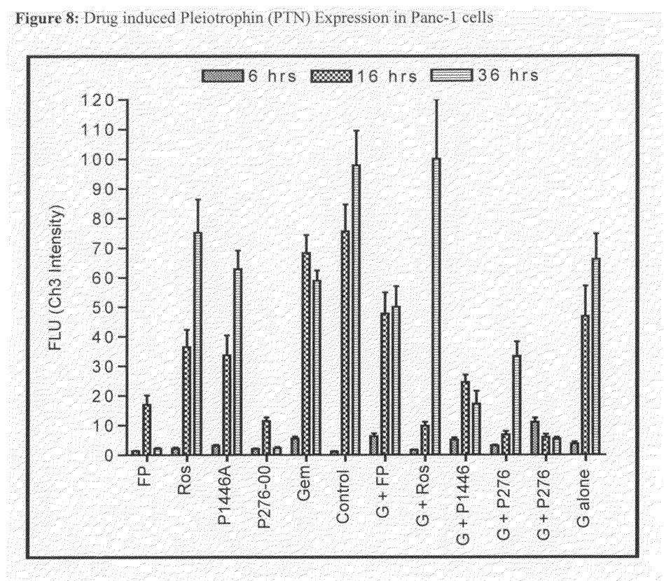

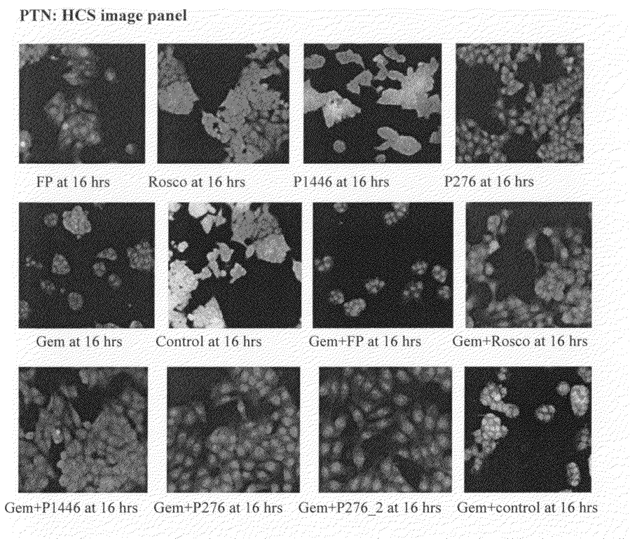

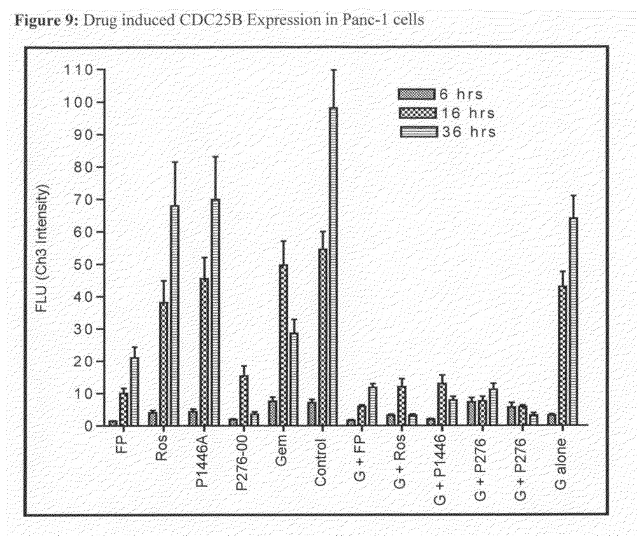

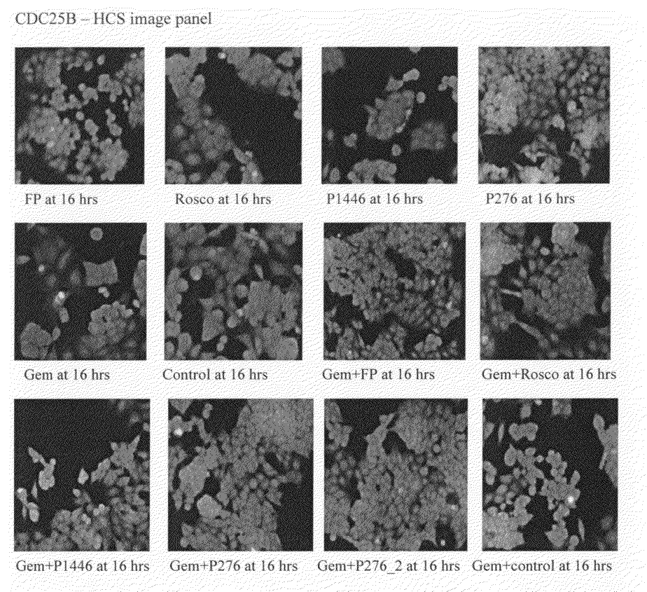

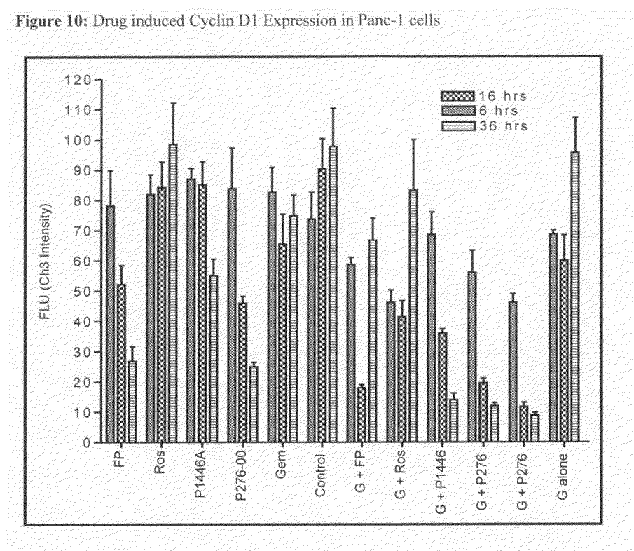

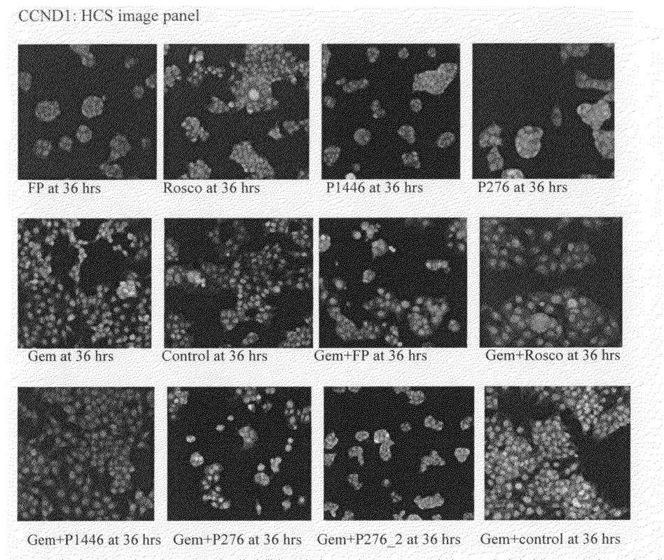

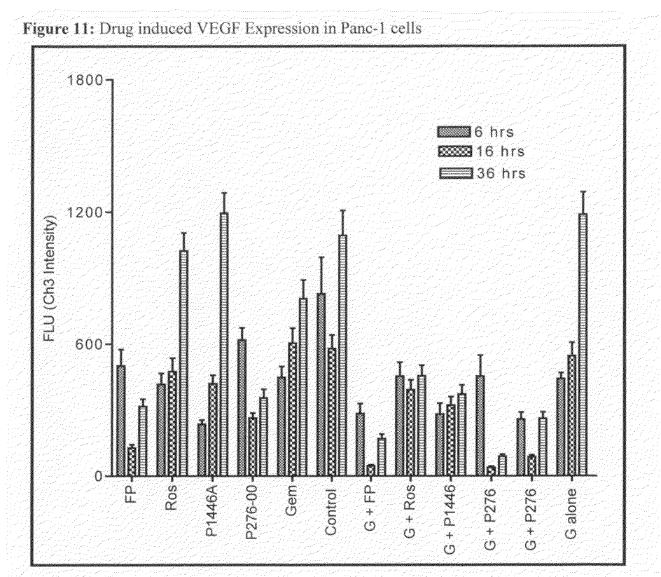

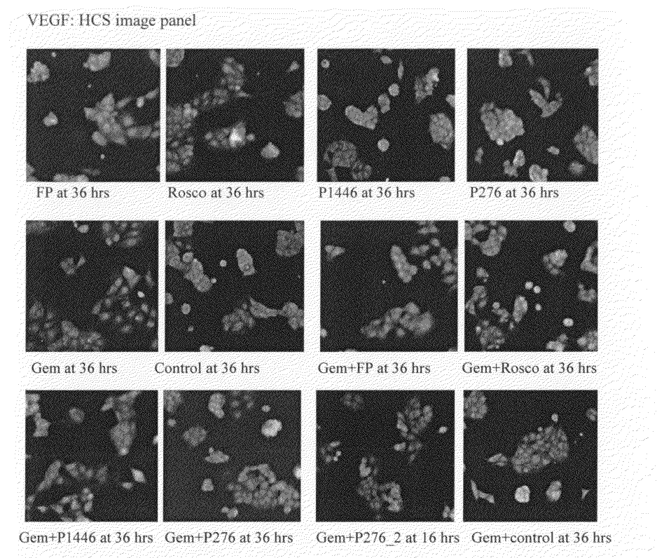

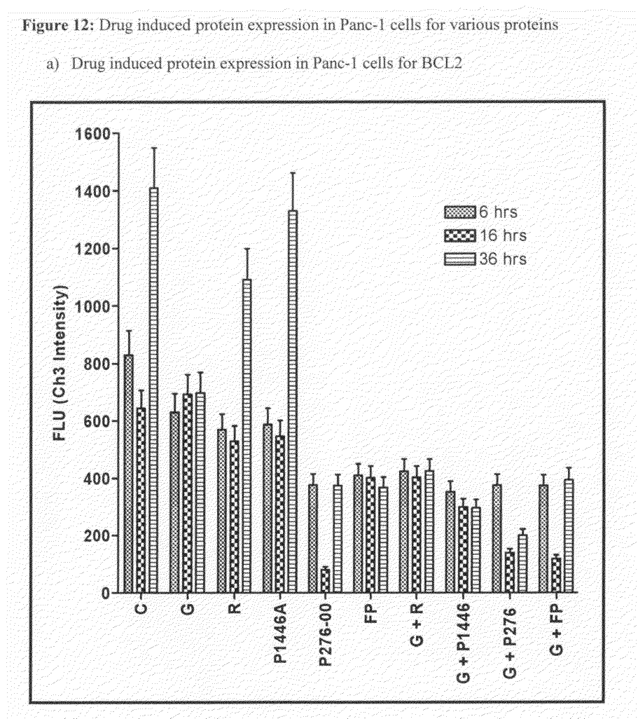

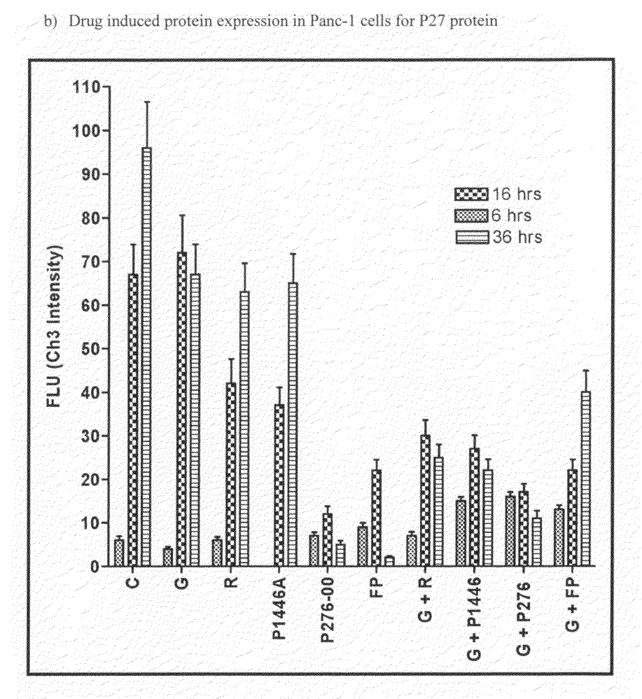

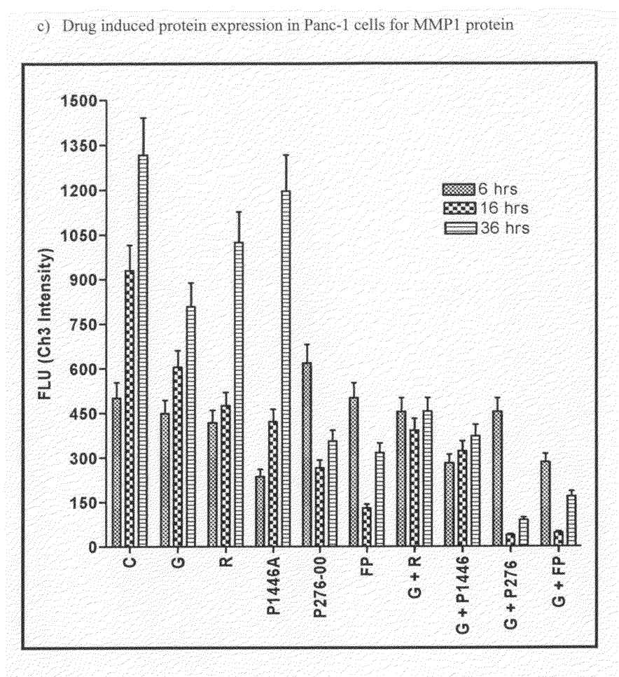

Results: It was observed that pAKT protein which is involved in cell proliferation and has cancer enhancing properties was significantly down-regulated during drug combination as compared to individual drug treatment (FIG. 6). Similarly protein expression levels of series of oncogenic proteins such as PTN, pRB, CDC25B, CCND1, VEGF, P27, BCL2, MMP1, COX2 and ID1 which facilitate tumor growth were significantly down-regulated during combination therapy as compared to individual drug exposure. On the other hand anti-tumor proteins such as CASP3 and GADD45-α levels were selectively up-regulated during combination of drug exposure as compared to individual drug therapy. It was also observed that PTN and GADD45-α which are established pancreatic cancer markers were selectively down-regulated by combination of gemcitabine and P276-00 as compared to other combinations (See Table 7 and FIGS. 7-12).

TABLE 7
Comparative analysis of protein expression measured as fluorescence units using cell-
imaging system at various time points after drug or drug combination exposure

| | | | | | | | |
|---|---|---|---|---|---|---|---|
| pRB | CASP3 | CCND1 | VEGF | COX2 | CDKN2A | CDC25B | BAX |

Protein ecpression 6 hours post treatment

| | | | | | | | | |
|---|---|---|---|---|---|---|---|---|
| Control | 1240 | 147 | 74 | 829 | 635 | 8 | 7 | 819 |
| G | 1318 | 115 | 83 | 448 | 462 | 60 | 8 | 892 |
| R | 1578 | 217 | 82 | 417 | 542 | 10 | 4 | 729 |
| P1446A | 1489 | 102 | 87 | 235 | 457 | 49 | 4 | 421 |
| P276-00 | 1540 | 152 | 84 | 618 | 406 | 76 | 2 | 700 |
| FP | 1523 | 111 | 78 | 501 | 351 | 67 | 1 | 481 |
| G + R | 1715 | 247 | 46 | 454 | 478 | 55 | 3 | 679 |
| G + P1446 | 1584 | 244 | 69 | 281 | 473 | 75 | 2 | 535 |
| G + P276 | 1668 | 249 | 46 | 259 | 405 | 58 | 6 | 707 |
| G + FP | 1737 | 141 | 59 | 284 | 463 | 64 | 2 | 497 |

Protein ecpression 16 hours post treatment

| | | | | | | | | |
|---|---|---|---|---|---|---|---|---|
| Control | 2583 | 202 | 90 | 579 | 1109 | 64 | 54 | 711 |
| Gem | 1931 | 570 | 66 | 604 | 1151 | 57 | 50 | 926 |
| Ros | 1632 | 1272 | 84 | 475 | 990 | 47 | 38 | 747 |
| P1446A | 1721 | 1157 | 85 | 420 | 1063 | 55 | 45 | 688 |
| P276-00 | 1400 | 607 | 46 | 264 | 375 | 9 | 15 | 644 |
| FP | 1300 | 536 | 52 | 129 | 466 | 14 | 10 | 252 |
| G + Ros | 1100 | 702 | 41 | 391 | 538 | 19 | 12 | 713 |
| G + P1446 | 822 | 499 | 36 | 322 | 367 | 14 | 13 | 633 |
| G + P276 | 951 | 635 | 20 | 38 | 298 | 6 | 8 | 367 |
| G + FP | 800 | 1196 | 18 | 46 | 264 | 8 | 6 | 455 |

Protein ecpression 36 hours post treatment

| | | | | | | | | |
|---|---|---|---|---|---|---|---|---|
| Control | 2840 | 265 | 98 | 1093 | 86 | 1441 | 98 | 715 |
| Gem | 2188 | 633 | 75 | 807 | 55 | 1483 | 29 | 1783 |
| Ros | 1889 | 1335 | 98 | 1024 | 67 | 1322 | 68 | 1785 |
| P1446A | 1978 | 1220 | 55 | 1195 | 43 | 1395 | 70 | 1670 |
| P276-00 | 1657 | 670 | 25 | 355 | | 707 | 4 | 1120 |
| FP | 1557 | 599 | 27 | 316 | 2 | 798 | 21 | 1049 |
| G + FP | 1057 | 1259 | 67 | 169 | 13 | 596 | 12 | 1709 |
| G + Ros | 1357 | 765 | 83 | 455 | 21 | 870 | 3 | 1215 |
| G + P1446 | 1079 | 562 | 14 | 372 | 17 | 699 | 8 | 1012 |
| G + P276 | 1208 | 698 | 12 | 89 | 12 | 630 | 11 | 1148 |

| | | | | | | | |
|---|---|---|---|---|---|---|---|
| ID1 | MMP-1 | P27 | BCL2 | GADD45-α | COX15 | PTN | GAPDH |

Protein ecpression 6 hours post treatment

| | | | | | | | | |
|---|---|---|---|---|---|---|---|---|
| Control | 3 | 501 | 6 | 830 | 3 | 627 | 1 | 80 |
| G | 7 | 448 | 4 | 630 | 4 | 1100 | 6 | 84 |
| R | 16 | 417 | 6 | 569 | 2 | 936 | 2 | 81 |
| P1446A | 38 | 235 | 4 | 587 | 3 | 628 | 3 | 78 |
| P276-00 | 1 | 618 | 7 | 376 | 10 | 907 | 2 | 96 |
| FP | 2 | 501 | 9 | 410 | 1 | 689 | 1 | 80 |
| G + R | 8 | 454 | 7 | 424 | 9 | 886 | 2 | 94 |
| G + P1446 | 15 | 281 | 15 | 353 | 4 | 742 | 5 | 93 |
| G + P276 | 38 | 259 | 16 | 305 | 39 | 914 | 11 | 89 |
| G + FP | 2 | 284 | 13 | 374 | 3 | 704 | 6 | 90 |

Protein ecpression 16 hours post treatment

| | | | | | | | | |
|---|---|---|---|---|---|---|---|---|
| Control | 77 | 929 | 67 | 644 | 13 | 718 | 76 | 100 |
| Gem | 62 | 604 | 72 | 693 | 70 | 1134 | 68 | 98 |
| Ros | 55 | 475 | 42 | 529 | 49 | 954 | 37 | 99 |
| P1446A | 67 | 420 | 37 | 546 | 46 | 896 | 34 | 100 |
| P276-00 | 18 | 264 | 12 | 81 | 23 | 852 | 12 | 100 |
| FP | 19 | 129 | 22 | 401 | 15 | 459 | 17 | 100 |
| G + Ros | 19 | 391 | 30 | 402 | 15 | 920 | 10 | 100 |
| G + P1446 | 11 | 322 | 27 | 299 | 17 | 841 | 25 | 100 |
| G + P276 | 12 | 38 | 16 | 139 | 48 | 574 | 7 | 96 |
| G + FP | 5 | 46 | 22 | 119 | 24 | 662 | 48 | 98 |

Protein ecpression 36 hours post treatment

| | | | | | | | | |
|---|---|---|---|---|---|---|---|---|
| Control | 98 | 1316 | 96 | 1410 | 19 | 722 | 100 | 98 |
| Gem | 26 | 807 | 67 | 698 | 51 | 1990 | 94 | 59 |
| Ros | 56 | 1024 | 63 | 1091 | 44 | 1992 | 98 | 75 |
| P1446A | 83 | 1195 | 65 | 1330 | 43 | 1878 | 99 | 63 |
| P276-00 | 6 | 355 | 5 | 374 | 31 | 1327 | 100 | 2 |
| FP | 3 | 316 | 2 | 367 | 5 | 1256 | 100 | 2 |
| G + FP | 3 | 169 | 40 | 394 | 33 | 1916 | 91 | 50 |
| G + Ros | 21 | 455 | 25 | 425 | 16 | 1422 | 100 | 100 |
| G + P1446 | 37 | 372 | 22 | 296 | 35 | 1219 | 92 | 17 |
| G + P276 | 9 | 89 | 19 | 200 | 50 | 1355 | 100 | 33 |

G, Gem = Gemcitabine, R, Ros = Roscovitine, FP = Flavopiridol

Expression of proteins listed in Table 7 were determined in terms of fluorescence unit obtained by using target activation algorithm from High content imaging system from Cellomics.
Protein expression data expressed as fluorescent units from Table 7 were used to generate expression ratio by comparing drug treatments (individual or combination) against the normal control with no drug treatment. The ratio of expression which is considered as dys-regulation (1.5 fold above or below the range for up-regulation and down regulation respectively) is subtracted from the normal range (±1.5) and is used to measure the efficacy score.

Expression ratio=protein expression during drug treatment/protein expression under normal control.

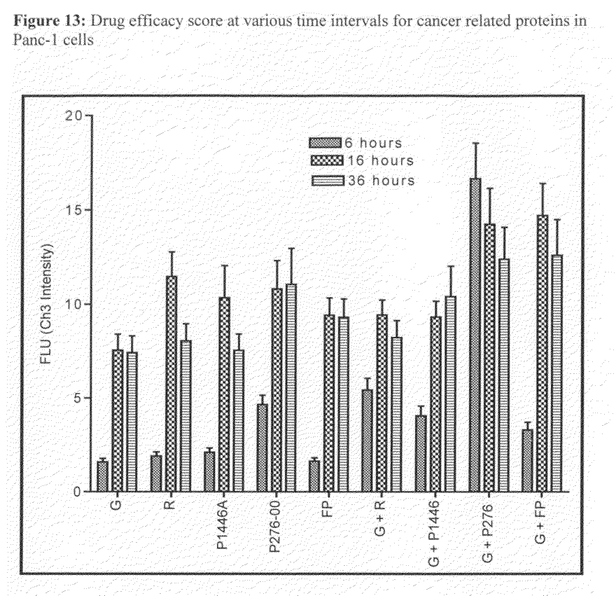

Efficacy score =Cumulative score (expression ratio−±1.50) for all proteins. Table 8 and FIG. 13 show the cumulative efficacy score for various proteins as shown in Table 7 calculated at time points 6, 16 and 36 hours drug exposure as a measure of drug efficacy. It was observed that best efficacy score is achieved for the combination of gemcitabine and P276-00 at 6 hours post exposure and is significantly higher than any of the individual drug or drug combinations clearly indicating the target specific effect of the combination of gemcitabine and P276-00. It is likely that almost similar efficacy score observed at later time points for flavopiridol is due to prolonged exposure of the drug and resultant cytotoxic effect rather than target specific effect.

TABLE 8
Efficacy score determination

Efficacy score

| Drugs | 6 hours | 16 hours | 36 hours |
|---|---|---|---|
| Gemcitabine | 1.6 | 7.6 | 7.4 |
| Roscovitine | 1.9 | 11.5 | 8.1 |
| P1446A | 2.1 | 10.3 | 7.6 |
| P276-00 | 4.7 | 10.8 | 11.1 |
| Flavopiridol | 1.6 | 9.4 | 9.3 |
| Gemcitabine + Roscovitine | 5.4 | 9.4 | 8.2 |
| Gemcitabine + P1446A | 4.1 | 9.3 | 10.4 |
| Gemcitabine + P276-00 | 16.7 | 14.2 | 12.4 |
| Gemcitabine + Flavopiridol | 3.3 | 14.7 | 12.6 |

We claim:

1. A gene signature comprising at least two drug response markers for monitoring drug response in a cancer patient administered with a combination of Gemcitabine and P276-00.

2. The gene signature of claim 1, wherein the drug response markers are selected from the group consisting of SNX7, FA38A, DNAI1, RRM2, CDK8, DLG5, FGF5, MKKS, HELLS, PPIL4, SLC19A2, ID1, DICER1, TMPRSS3 and HIST1H2BO, BAX, Cytochrome C, Caspase-3, pAKT, pRB, CyclinD1, MMP-1, VEGF, CDC25B, P21, Pl4ARF and PTN.

3. The gene signature of claim 2, wherein the drug response markers SNX7, FA38A, DNAI1, BAX, Cytochrome C and Caspase-3 are up regulated in the cancer patient administered with the combination of Gemcitabine and P276-00.

4. The gene signature of claim 2, wherein the drug response markers RRM2, CDK8, DLG5, FGF5, MKKS, HELLS, PPIL4, SLC19A2, ID1, DICER1, TMPRSS3, HIST1H2BO, pAKT, pRB, CyclinD1, MMP-1, VEGF, CDC25B, P21, P14ARF and PTN are down regulated in the cancer patient administered with the combination of Gemcitabine and P276-00.

5. A gene signature comprising at least two drug response markers for monitoring drug response in a cancer patient administered with a combination of Gemcitabine and P1446A.

6. The gene signature of claim 5, wherein the drug response markers are selected from the group consisting of P21, REV3L, FGF5, PTK7, POLH, P27 and SSTR2.

7. The gene signature of claim 5, wherein the markers P21, REV3L, FGF5, PTK7, POLH, P27 and SSTR2 are up regulated in the cancer patient administered with the combination of Gemcitabine and P1446A.

8. A method of monitoring drug response in a patient suffering from cancer treated with a combination of Gemcitabine and P276-00, comprising detection of a gene signature with at least two drug response markers.

9. The method of claim 8, wherein the drug response markers are selected from the group consisting of SNX7, FA38A, DNAI1, RRM2, CDK8, DLG5, FGF5, MKKS, HELLS, PPIL4, SLC19A2, D1, DICER1, TMPRSS3, HIST1H2BO, BAX, Cytochrome C, Caspase-3, pAKT, pRB, CyclinD1, MMP-1, VEGF, CDC25B, P21, P14ARF and PTN.

10. A method of monitoring drug response in a patient suffering from cancer treated with a combination of Gemcitabine and P1446A, comprising detection of a gene signature with at least two drug response markers.

11. The method of claim 10, wherein the drug response markers are selected from the group consisting of P21, REV3L, FGF5, PTK7, POLH, P27 and SSTR2.
